(19) United States
(12) Patent Application Publication
(10) Pub. No.: US 2016/0120593 A1
(43) Pub. Date: May 5, 2016

(54) THERMALLY TREATING TORN TISSUE

(71) Applicant: EMPIRE TECHNOLOGY DEVELOPMENT LLC, Wilmington, DE (US)

(72) Inventors: Trevor John MOODY, Seattle, WA (US); Scott WOLF, Menlo Park, CA (US)

(21) Appl. No.: 14/892,525

(22) PCT Filed: Apr. 21, 2014
(86) PCT No.: PCT/US14/34829

§ 371 Date: Nov. 19, 2015

(51) Int. Cl. A61B 18/14

(52) U.S. Cl. CPC A61B 18/148; A61B 2018/0022

(57) ABSTRACT

An apparatus for thermally treating torn tissue includes a cannula, a balloon, and one or more electrically conductive electrodes. The cannula includes a hollow interior that is configured to receive a fluid. At least a portion of the balloon is positioned within the hollow interior of the cannula, and fluid received through the hollow interior of the cannula inflates the balloon. The one or more electrically conductive electrodes are mounted to the balloon and are configured to deliver heat to tissue.

### BACKGROUND

The following description is provided to assist the understanding of the reader. None of the information provided or references cited is admitted to be prior art.

Knee pain caused by osteoarthritis, other diseases, or trauma such as tissue tears affects tens of millions of people in the United States. By 85 years of age, approximately 50% of all people will experience knee pain caused by tissue tears or osteoarthritis of the knee. After the disease has significantly progressed, effective but expensive and highly invasive knee joint replacements are available. However, in the earlier stages of the disease, a limited range of therapies are available. Injection of hyaluronic acid is commonly performed and can provide some temporary pain relief. Unfortunately, no early therapy has demonstrated extended pain relief, any impact on progression of the disease, or an ability to delay the need for a total joint replacement.

One cause of progressive osteoarthritis is meniscal tears. The natural course of cartilage loss also appears to be accelerated in the presence of meniscal tears. There is a strong relation between meniscal tears and lesions that have progressed more rapidly, and meniscal abnormalities are known to have led to enhanced chondromalacia as a result of abnormal articular forces. Photoelastic studies have shown that the meniscus serves to protect articular cartilage by distributing load throughout the articular surface and preventing focal stress concentrations.

Problems with the knee meniscus and tissues are often a frequent source of knee pain on their own. Removal of the meniscus is an extremely common procedure performed by orthopaedic surgeons within the United States. This is true despite the understanding that partial or complete menisectomy is strongly associated with more rapid development of osteoarthritis.

### SUMMARY

An illustrative apparatus includes a cannula, a balloon, and one or more electrically conductive electrodes. The cannula includes a hollow interior that is configured to receive a fluid. At least a portion of the balloon is positioned within the hollow interior of the cannula, and fluid received through the hollow interior of the cannula inflates the balloon. The one or more electrically conductive electrodes are mounted to the balloon and are configured to deliver heat to tissue.

An illustrative method for thermally welding torn tissue includes inserting at least a portion of a cannula into an intra-articular space. The cannula includes a hollow interior. A balloon is inflated within the intra-articular space such that one or more electrically conductive electrodes mounted to the balloon contact tissue. Heat is delivered to the tissue through the one or more electrically conductive electrodes.

An illustrative method of creating an apparatus to treat torn tissue includes forming a cannula that includes a hollow interior, coupling one or more electrically conductive electrodes to a balloon, coupling at least a portion of the balloon to the hollow interior of the cannula, and coupling conductive wiring to the one or more electrically conductive electrodes.

An illustrative system includes an apparatus to treat torn tissue and a computing device. The apparatus includes a cannula having a hollow interior, a balloon configured to be deployed through a distal end of the hollow interior of the cannula, one or more electrically conductive electrodes coupled to the balloon and configured to deliver heat to tissue, and a sensor coupled to the one or more electrically conductive electrodes. The computing device includes a memory configured to receive and store temperature feedback information from the sensor, and a processor operatively coupled to the memory and configured to control heat output of the one or more electrically conductive electrodes based on the temperature feedback information.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 is a diagram illustrating the general anatomy of a human knee.

FIG. 2 is a diagram illustrating an apparatus for thermally treating torn tissue in accordance with an illustrative embodiment.

FIG. 3 is a diagram illustrating an apparatus for thermally treating torn tissue in accordance with an illustrative embodiment.

FIG. 4 is a diagram illustrating an apparatus being used to thermally treat torn tissue in accordance with an illustrative embodiment.

FIG. 5 is a diagram illustrating an apparatus being used to thermally treat torn tissue in accordance with an illustrative embodiment.

FIG. 6 is a flow diagram illustrating a process for thermally welding torn tissue in accordance with an illustrative embodiment.

FIG. 7 is a flow diagram illustrating a process for creating an apparatus to treat torn tissue in accordance with an illustrative embodiment.

FIG. 8 is a diagram illustrating a system for thermally treating torn tissue in accordance with an illustrative embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

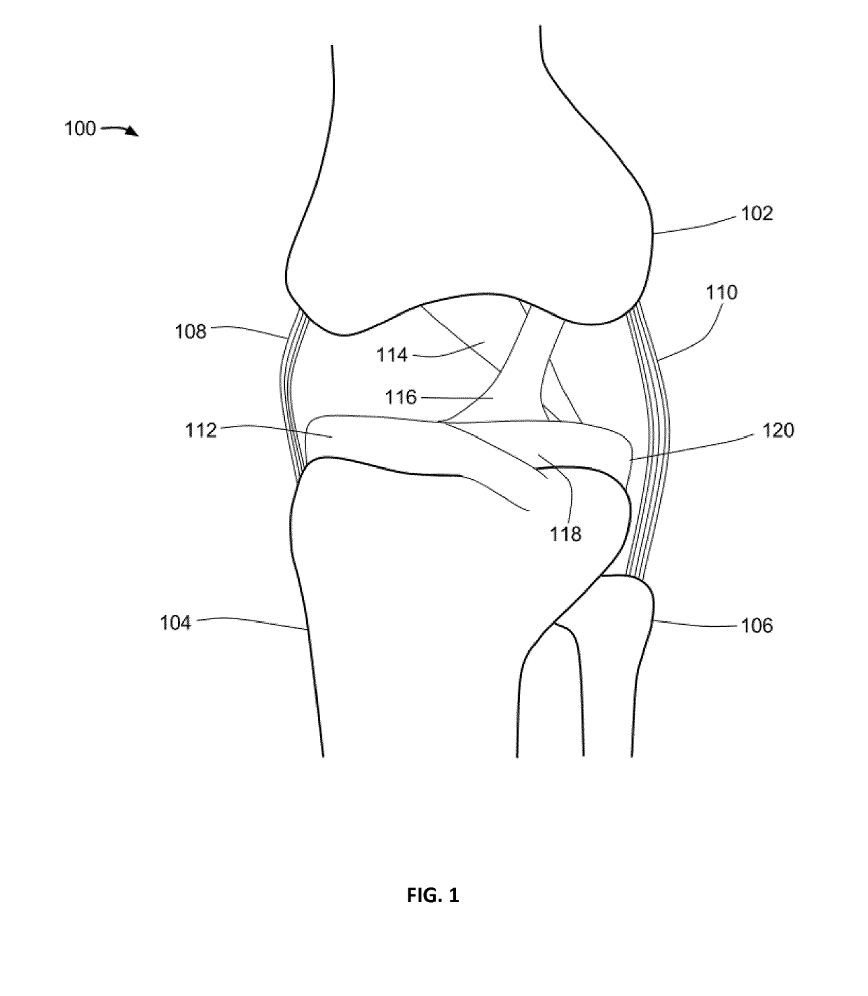

FIG. 1 illustrates the general anatomy of a human knee 100. Human knee 100 includes a femur 102, a tibia 104, a fibula 106, a medial collateral ligament 108, a lateral collateral ligament 110, a medial meniscus 112, a posterior cruciate ligament 114, an anterior cruciate ligament 116, a transverse ligament 118, and a lateral meniscus 120. The primary embodiments described herein are discussed with respect to treatment of torn meniscal tissue in a human (e.g., a torn medial meniscus, a torn lateral meniscus, etc.) through insertion into the intra-articular space of a knee. However, it should be understood that other embodiments herein can be used to treat other types of torn tissue in a human. Further, the scope of the present application is not limited to the treatment of a human, but may also be used in the treatment of animals such as dogs, cats, cows, horses, etc.

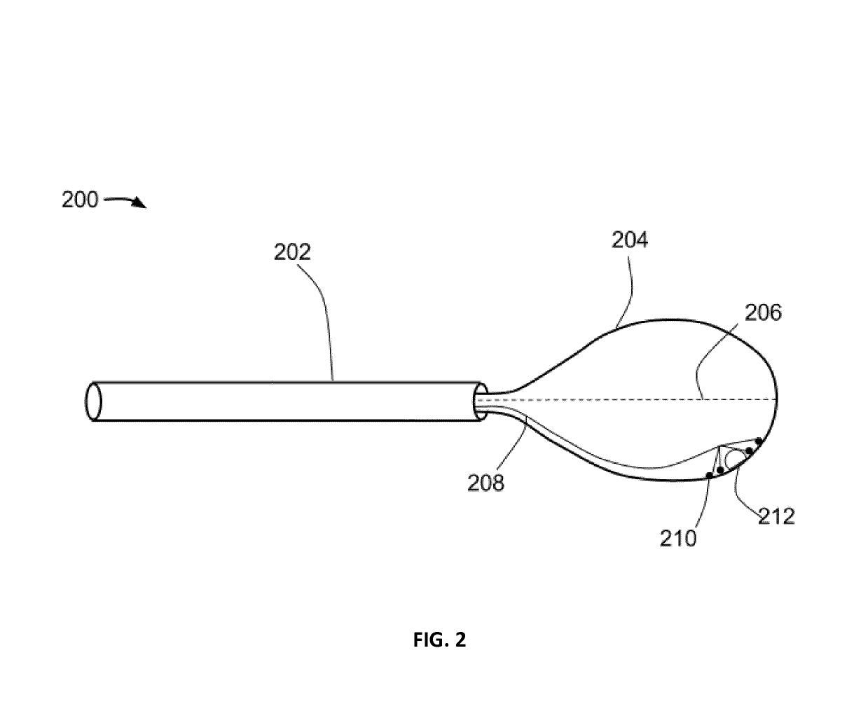

FIG. 2 is an apparatus 200 for thermally treating torn tissue in accordance with an illustrative embodiment. Apparatus 200 includes a cannula 202 with a hollow interior portion, a balloon 204, a guidewire 206, conductive wiring 208, electrodes 210, and a thermistor 212. In alternative embodiments, apparatus 200 may include fewer, additional, and/or different components. In an illustrative embodiment, cannula 202 is the cannula of a needle. Accordingly, cannula 202 may have a pointed, sharp end for puncturing. The pointed end may be beveled to create a sharp pointed tip. In this manner, cannula 202 may deliver balloon 204 laterally into the intra-articular space of the knee in similar fashion to needles that are commonly placed laterally into the intra-articular space of the knee to deliver hyaluronic acid or cortecosteroids. In another embodiment, cannula 202 may be part of a trocar (or trocar-like) device utilized for minimally invasive delivery of apparatus 200.

Cannula 202 may have a length that is defined by the particular anatomy of the patient. The intra-articular space of the knee varies in dimensions from patient to patient. As such, cannula 202 may be longer for a patient having a larger intra-articular space, and smaller for a patient having a smaller intra-articular space. In this manner, a clinician may have a variety of apparatuses 200 with different cannula 202 configurations, and may select an apparatus 200 including the appropriate length cannula 202 for the patient. The length of cannula 202 may also be defined by a clinician's handling preferences. Typically, the length of cannula 202 will range from 15-30 cm, however, other cannula 202 lengths are also envisioned. The diameter of cannula 202 may also be defined by the particular anatomy of the patient or clinician handling preferences. For example, a patient having a larger intra-articular space may warrant a cannula with a larger diameter in order to deploy a larger balloon 204. Typically, the inner diameter of cannula 202 will range from 1-5 mm, however, other cannula 202 diameters are also envisioned. Cannula 202 may be constructed from any rigid biocompatible material. As an example, this may include a biocompatible metal, stainless steel, Titanium, Nitinol, biocompatible plastic, and the like. Balloon 204 may be constructed from any highly flexible biocompatible material, and may have overall dimensions that are defined by the particular anatomy of the patient. In one embodiment, balloon 204 is elliptical through its cross section and has major and minor semi-axes defined by the intra-articular space of a particular patient. An example elliptical balloon 204 has a length (major axis) of 40 mm, a width (minor axis) of 35 mm, and height of 25 mm, although other dimensions may be used. In another embodiment, balloon 204 is spherical through its cross section. In another embodiment, balloon 204 is “saucer” shaped. In another embodiment, balloon 204 is “football” shaped. Balloon 204 may be constructed from a variety of materials. As an example, balloon 204 may comprise a polymer (e.g., polyimide, polyethylene terephthalate (PET)), a mixture of polymers and elastomers, latex, silicone, polyvinyl chloride, cross-linked nylon, or polyurethane.

Balloon 204 may be mounted to cannula 202 in a variety of ways. In one embodiment, a portion of balloon 204 is positioned within the hollow interior of cannula 202, and at least a portion of balloon 204 is coupled to cannula 202. In another embodiment, the opening of balloon 204 is permanently fixed to the distal end of cannula 202. In such an arrangement, balloon 204 is sealed to cannula 202 such that fluid used to inflate balloon 204 does not escape from the interior of balloon 204 and the interior of cannula 202. Balloon 204 may be mounted to cannula and a seal formed by using an adhesive, chemical bonding, or thermal bonding. Further, in this arrangement balloon 204 may be positioned at least partially within cannula 202, and the body of balloon 204 may be deployed as balloon 204 is inflated. Prior to deployment and inflation, balloon 204 may be fitted within cannula 202 via folding, rolling, etc. In another embodiment, balloon 204 is not permanently fixed to cannula 202, but is instead delivered through cannula 202 using guidewire 206 and is then inflated. In this embodiment, balloon 204 may include a rigid ring attached to its opening, which may couple to the distal end of cannula 202 or otherwise create a seal as balloon 204 is deployed. It should be noted that an embodiment may make use of a multitude of the discussed mounting configurations.

Balloon 204 may be delivered through cannula 202 into an intra-articular space, such as the intra-articular space of a knee that is adjacent to meniscal tissue. Alternatively, balloon 204 may be delivered to any other intra-articular space to treat tissue as described herein. Guidewire 206 may be used to deliver balloon 204 through cannula 202 and provide mechanical support to balloon 204 after it is deployed. Guidewire 206 may be flexible or sufficiently rigid to allow guidewire 206 to push balloon 204 through cannula 202. Guidewire 206 may be constructed from various materials, including stainless steel, titanium, and Nitinol, and may be of a gauge corresponding to the dimensions of cannula 202. Typically, guidewire 206 will range in outer diameter from 0.5 mm to 1.5 mm, although other diameters are envisioned. In one embodiment, guidewire 206 is coupled to the interior of balloon 204. Guidewire 206 may be coupled to the interior of balloon 204 using adhesive, chemical bonding, or thermal bonding. In one embodiment, the tip of guidewire 206 is coupled (e.g., welded) to a small metal ring embedded in the material of balloon 204. In this manner, guidewire 206 may be also used to retract and pull balloon 204 into cannula 202 after use. In another embodiment, guidewire 206 is not affixed to balloon 204 and may be removed after balloon 204 is deployed. In this embodiment suction forces applied to apparatus 200 through cannula 202 can be used to retract balloon 204. The negative pressure from suction forces can cause balloon 204 to withdraw within cannula 202. Although in FIG. 2 guidewire 206 is depicted as a single wire, alternative embodiments are envisioned. For example, guidewire 206 may have a branched end such that a rounded structure is formed within balloon 204 to provided additional support. As another example, guidewire 206 may contain two or more wires which may be independently adjusted or controlled as balloon 204 is deployed.

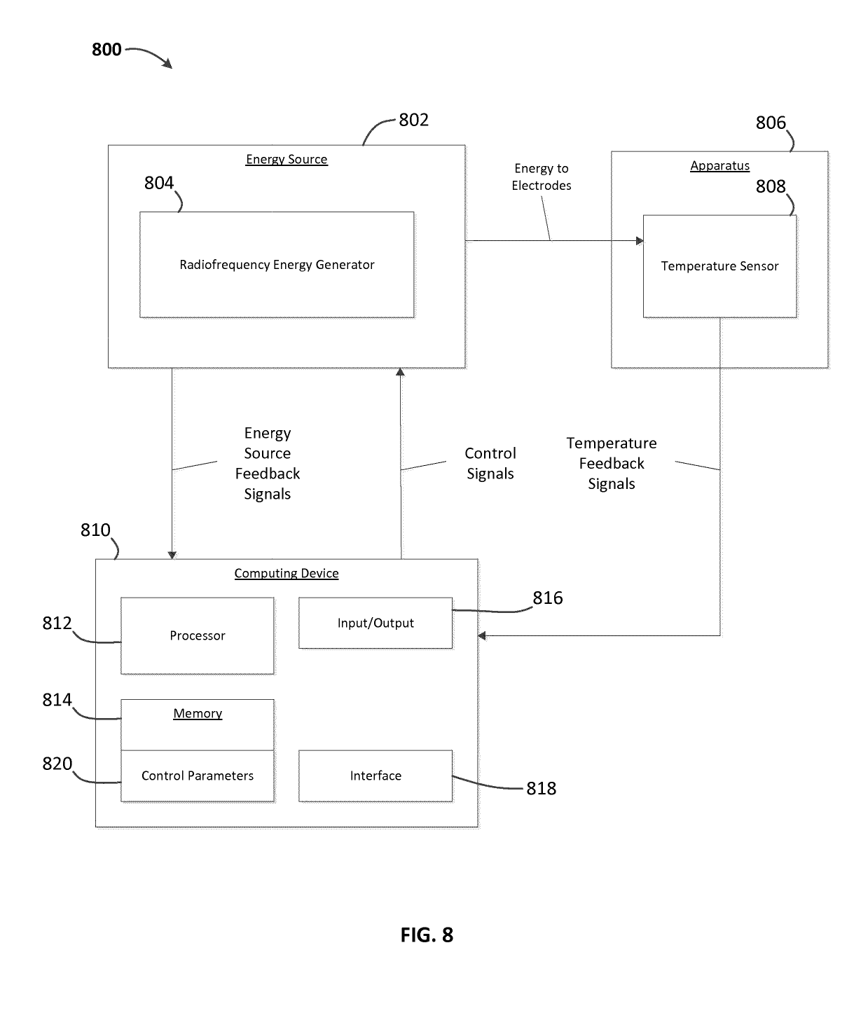

After deployment, balloon 204 may be inflated by fluid provided through cannula 202. The fluid used to inflate balloon 204 may be gaseous (e.g., helium, carbon dioxide, etc.) or a liquid (e.g., a sterile saline solution, radio-opaque liquid, etc.) as known to those skilled in the art. In one embodiment, a volume-limited syringe is used to deliver a specific/measured amount of inflation fluid in order to inflate balloon 204. In this manner, a clinician can know that balloon 204 is sufficiently inflated when the delivery syringe is empty. Alternatively, the delivery device or syringe may include a pressure gauge that the clinician may assess when delivering the inflation fluid. When a desired pressure is reached, the clinician may determine that balloon 204 is sufficiently inflated. In another embodiment, an external pump may be utilized to deliver the inflation fluid through cannula 202 to balloon 204. The external pump may contain a pressure sensing device used to monitor the inflation process and pressure of the balloon. Delivery of fluid may also be automated and controlled by a computing device (e.g., computer 810 of FIG. 8), or may be manually controlled by a clinician. The amount of fluid to be delivered may depend on the size of balloon 204 or a volume (i.e. an intra-articular space) to be filled by balloon 204. Delivery of fluid may be stopped when a desired volume or pressure has been reached. The computing device may accept data from a pump or other fluid delivery means in order to monitor the pressure and amount of fluid used during deployment and inflation. The computing device may provide this information to a clinician via a display.

In one embodiment, apparatus 200 does not contain a guidewire. In this embodiment, the fluid disposed through cannula 202 in order to deploy and inflate balloon 204 may also provide structural support. The fluid may remain disposed within balloon 204 and pressurized during deployment and use. The fluid may then be removed from balloon 204 and cannula 202 using suction means (e.g., a pump) attached to apparatus 200. Such suction forces may remove fluid and cause balloon 204 to retract within cannula 202 due to negative pressure created during suction.

Electrodes 210 mounted to balloon 204 are arranged such that when balloon 204 is inflated within the intra-articular space, electrodes 210 may contact torn tissue (e.g., a meniscal tear) that is adjacent to the intra-articular space. Generally, electrodes 210 are mounted to the distal end of the exterior of balloon 204 in order to maximize electrode contact with the meniscus. Electrodes 210 may be mounted using a biocompatible adhesive, and balloon 204 may be maximally inflated during the mounting process. Electrodes 210 may be constructed from metal or alloys with sufficient conductive properties as is known to those of skill in the art. For example, conductors of electrodes 210 may comprise gold (Au), chromium/gold alloy (Cr/Au), etc. Electrodes 210 may contain one or more electrode devices. The size, shape, position, and other characteristics of electrodes 210 may be selected in order to create a heated area with specific properties. Specific properties of the heat delivery area may include size, shape, depth, and temperature gradient. As an example, the size of a heat delivery area can be increased or decreased corresponding to the distance between each of the electrodes 210. As another example, the shape of the heat delivery area directly corresponds to the mounting pattern of electrodes 210. A circular heating area may utilize electrodes 210 in a circular mounting pattern. A linear heat delivery area may utilize a linear arrangement of electrodes 210. As another example, the depth of a heat delivery may correspond to the density of electrodes 210 on balloon 204.

Electrodes 210 are arranged such that a current provided by a radiofrequency energy generator flows through tissue between each pair (i.e. an anode and cathode arrangement). The current flows through the electrodes such that radio frequency (RF) energy radiates out from the surface of the electrodes. The radiated RF energy heats the tissue areas in the radiated RF energy field. In one embodiment, the polarities of the electrodes may be such that one electrode of a pair serves to deliver energy, and the other electrode of the pair serves to return energy back to the energy source. In another embodiment, there may be a single electrode configured to serve as a ground, or return, electrode. In this manner current may flow from source electrodes through the ground electrode. The spacing of electrodes 210 may be selected to correspond to the size or length of a tear in tissue to be thermally welded. For example, a larger tear may utilize a balloon 204 with electrodes 210 that are comparatively further apart than a smaller tear would utilize. As another example, a precise temperature gradient across a certain distance may be utilized to weld a specific area of torn tissue. Electrodes 210 may be spaced on balloon 204 accordingly (i.e. for a larger distance temperature gradient, electrodes 210 may be spaced further apart as compared to a smaller distance temperature gradient). In practice, a clinician may have a variety of apparatuses with different electrode configurations, and can select a particular apparatus 200 for a particular patient application. For example, one embodiment includes electrodes 210 arranged for precisely targeted thermal welding. Another embodiment includes electrodes 210 arranged to facilitate a temperature increase (i.e. a temperature to warm the tissue but not hot enough to thermally weld the tissue) in order to stimulate the body's natural healing mechanisms.

A computing device (e.g., computer 810 of FIG. 8) can control the delivery of energy from an energy source (e.g., energy source 802 of FIG. 8) to each of electrodes 210. The computing device may further control the polarity of the electrodes. In this manner, the computing device may cause certain electrodes 210 to deliver energy and certain electrodes 210 to return energy. In another embodiment, the computing device selects different amounts of energy to be sent to each pair of electrodes 210. In this manner, each pair of electrodes 210 may create a heat delivery area with a particular size, shape, depth, and temperature gradient. In another embodiment, the computing device causes the same amount of energy to be delivered to all pairs of electrodes 210, and electrodes 210 are controlled in unison.

After balloon 204 is inflated, energy (e.g., alternating current energy) may be delivered to electrodes 210 via conductive wiring 208 and a clinician (or other operator) of apparatus 200 may position the electrodes 210 in contact with, the torn tissue (e.g., a meniscal tear). Any type of conductive material/metal may be used to construct conductive wiring 208. For example, conductive wiring 208 may comprise metal, copper, aluminum, stainless steel, etc. The delivered energy may be varied in frequency, power level, etc., in order to create different energy penetration characteristics of the radiated RF energy from the electrodes. During positioning, the clinician may make use of a prior imaging scan, such as a CT, MRI, X-Ray, or other scan type known to those of skill in the art. The clinician may also utilize ultrasound information provided by an ultrasound device, thereby allowing the clinician to view in real time the anatomy of the intra-articular space as the clinician positions electrodes 210. In one embodiment, the clinician positions balloon 204 without using a visualization device. In this manner, the clinician may rely on the dimensions and conformal nature of inflated balloon 204 within the intra-articular space to position balloon 204. The electrodes 210 cause torn tissue to heat upon receiving alternating current energy via conductive wiring 208 and delivering the energy to the torn tissue. The energy delivered from the electrodes 210 to the torn tissue may be adjusted such that it is a sufficient heat to facilitate thermal welding of the torn tissue. The heat delivered to the damaged tissue may also be used to facilitate a temperature increase in the tissue, thereby leading to a quicker repair of the damaged tissue through stimulation of the natural healing mechanisms and processes of the body.

In an illustrative embodiment, a clinician (or operator) of apparatus 200 may receive feedback from thermistor 212, which is configured to sense temperature information. For example, E333 mini medical thermistor from Quality Thermistor, Inc. may be used as thermistor 212. Alternatively, other thermistors may be used. Thermistor 212 may be mounted to the exterior of balloon 204 using a biocompatible adhesive, and balloon 204 may be maximally inflated during the mounting process. The leads of thermistor 212 may run along the same path as conductive wiring 208. The feedback provided may correspond to temperature of the tissue near the electrodes 210, or temperature conditions of the electrodes 210. Such feedback may be accepted by a processing device and converted into a readable format, and output on a display (e.g., a measure of degrees Celsius, a temperature vs. time chart, etc.). The feedback may also be input to a system responsible for controlling the energy provided through conductive wiring 208 to electrodes 210. It should be noted that use of other temperature sensing devices (e.g., a resistance temperature detector, etc.) in a similar manner as thermistor 212 is within the scope of the present disclosure. In one embodiment, the clinician or system may use the feedback to monitor the temperature and adjust energy provided to electrodes 210 such that the temperature of torn meniscal tissue is heated to approximately 62 degrees Celsius, but not greater than 69 degrees Celsius. Energy may be applied to the torn meniscal tissue occur for approximately 10 seconds to 120 seconds to facilitate welding of the tissue, although other amounts of time may be used. Other temperature profiles and energy application times are also envisioned. Temperature profiles and energy application times may also be based on the particular procedure being performed and/or the anatomy of the patient. Previous work in this field has shown that by heating torn meniscal tissue to approximately 62 degrees Celsius, it is possible to thermally weld together separated tissue even within the avascular “white-white” zone of the meniscus, which otherwise may be less amenable to repair because of inadequate vascularisation.

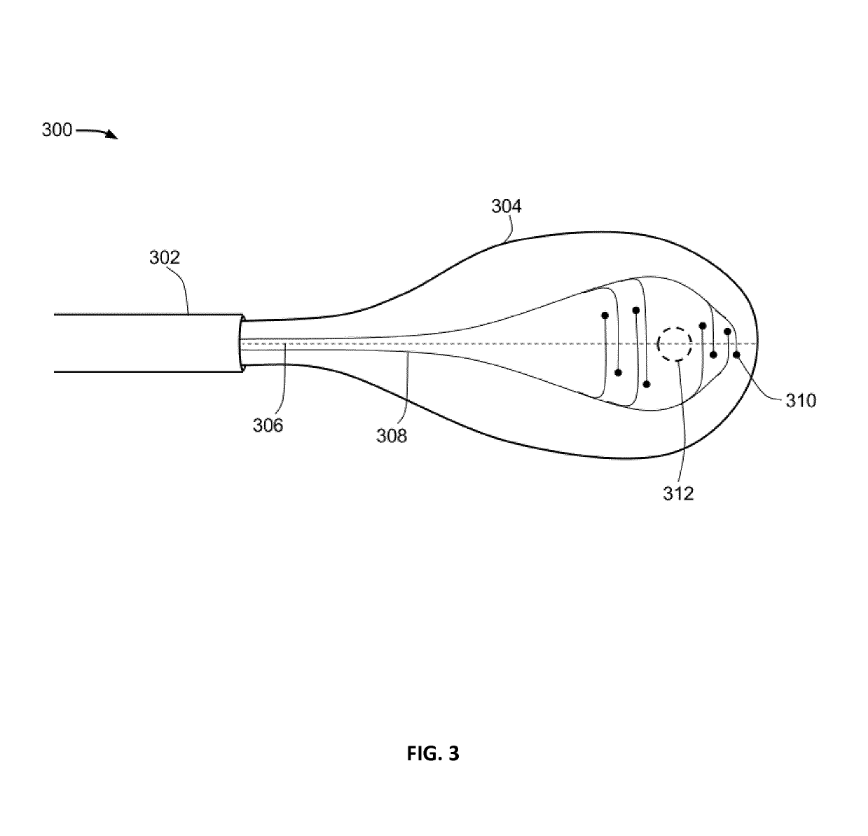

FIG. 3 illustrates an apparatus 300 for thermally treating torn tissue in accordance with an illustrative embodiment. Apparatus 300 may be an apparatus for thermally treating torn tissue as described herein (e.g., apparatus 200 of FIG. 2, etc.), shown in a planar view. Apparatus 300 includes a cannula 302, a balloon 304, a guidewire 306, conductive wiring 308, electrodes 310, and thermistor 312. Electrodes 310 are mounted to balloon 304 such that when balloon 304 is inflated within the intra-articular space, electrodes 310 may contact the torn tissue (e.g., a meniscal tear). FIG. 3 depicts an illustrative arrangement of electrodes 310 on balloon 304. As shown, the electrodes 310 are arranged in pairs, where one electrode of the pair delivers energy provided by an energy generator, and the other electrode in the pair returns energy back to the energy generator, allowing energy to flow therebetween. Such an arrangement may be defined according to the polarity of the electrodes 310. Temperature sensing thermistor 312 is mounted to the balloon 304 such that it may sense the temperature of the heated area created by electrodes 310. In another embodiment, electrodes 310 and thermistor 312 may be mounted to balloon 304 according to a mounting pattern different from that depicted in FIG. 3. It should be noted that the scope of the present application is not limited to a particular mounting pattern of electrodes 310 or thermistor 312 on balloon 304.

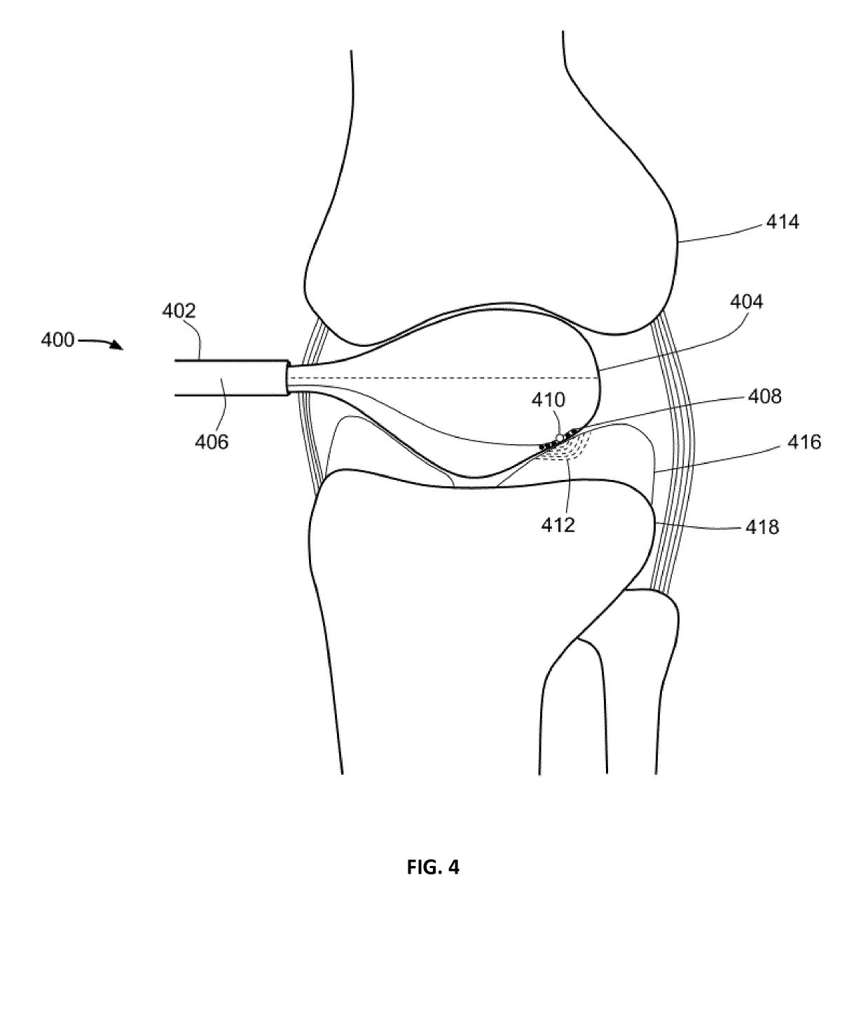

FIG. 4 illustrates an apparatus 400 being used to thermally treat torn tissue in accordance with an illustrative embodiment. Apparatus 400 may be an apparatus for thermally treating torn tissue as described herein (e.g., apparatus 200 of FIG. 2, apparatus 300 of FIG. 3, etc.). Apparatus 400 includes a cannula 402, a balloon 404, a guidewire 406, and conductive wiring coupled to electrodes 408. Guidewire 406 may be used to deliver balloon 404 through cannula 402 and provide mechanical support to balloon 404 after it is deployed. After deployment, balloon 404 may be inflated by a fluid provided through cannula 402. The fluid may be gaseous or a liquid. Electrodes 408 are mounted to balloon 404 such that when balloon 404 is inflated within the intra-articular space, electrodes 408 contact the torn tissue (e.g., torn meniscal tissue). Temperature sensing thermistor 410 is mounted to the balloon 404 such that it may sense the temperature of heated area 412 created by electrodes 408. Thermistor 410 may provide temperature feedback related to heated area 412 to a computing device. The computing device can have a graphical display such that a clinician utilizing apparatus 400 is able to view the temperature feedback and adjust the energy provided to electrodes 408, and as a result, control the heat delivered to heated area 412.

In this embodiment, apparatus 400 is depicted as being deployed within the intra-articular space in between the femur 414 and the tibial plateau 418. Balloon 404 is configured such that it is conformal to the intra-articular space when it is inflated. In this manner, electrodes 408 and thermistor 410 may be positioned in close proximity to a defect in the lateral meniscus 416. Heated area 412 is generated by electrodes 408 in order to heat a defect in the lateral meniscus 416 and thermally weld torn tissue. Thermal welding may be accomplished according to temperature profiles as discussed with respect to apparatus 200 of FIG. 2.

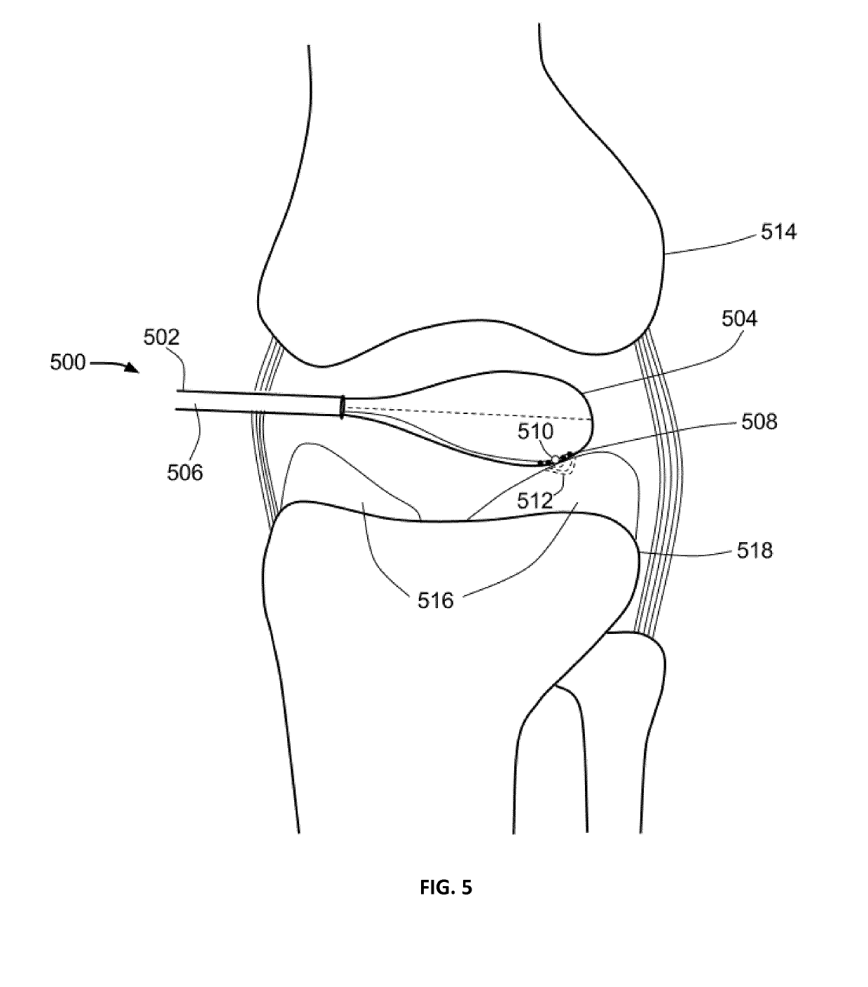

FIG. 5 illustrates an apparatus 500 being used to thermally treat torn tissue in accordance with an illustrative embodiment. Apparatus 500 may be an apparatus for thermally treating torn tissue as described herein (e.g., apparatus 200 of FIG. 2, apparatus 300 of FIG. 3, etc.). Apparatus 500 includes a cannula 502, a balloon 504, a guidewire 506, and conductive wiring coupled to electrodes 508. Guidewire 506 may be used to deliver balloon 504 through cannula 502 into an intra-articular space, and may provide mechanical support to balloon 504 after it is deployed. After deployment, balloon 504 may be inflated by a fluid provided through cannula 502. The fluid may be gaseous or a liquid. Electrodes 508 are mounted to balloon 504 such that when balloon 504 is inflated within the intra-articular space, electrodes 508 may contact the torn tissue (e.g., torn meniscal tissue). Temperature sensing thermistor 510 is mounted to the balloon 504 such that it may sense the temperature of heated area 512 created by electrodes 508. Thermistor 510 may provide temperature feedback related to heated area 512 to a computing device. The computing device can have a graphical display such that a clinician utilizing apparatus 500 is able to view the temperature feedback and adjust the energy provided to electrodes 508, and as a result, adjust heated area 512.

In this embodiment, apparatus 500 is depicted as being deployed within the intra-articular space in between the femur 514 and the tibial plateau 518. Balloon 504 is configured such that it is smaller than the intra-articular space when inflated (as compared to balloon 404 of FIG. 4, which is conformal to the intra-articular space when inflated). The size of the inflated balloon 504 may be controlled by an amount of fluid delivered to the balloon, or it may be a physical constraint of the dimensions of the balloon. In this manner, balloon 504 may be positioned such that electrodes 508 and thermistor 510 may be in close proximity to a defect in a range of different locations (e.g., medial meniscus and lateral meniscus 516, etc.) within the intra-articular space. This arrangement allows apparatus 500 to be used to treat multiple smaller tissue tears with a greater precision as compared to apparatus 400 of FIG. 4. In such an embodiment, balloon 504 may be of a size that is optimized for use during an arthroscopic procedure. This configuration is useful in targeting a specific area of damaged tissue. Arthroscopic visualization systems may also be used to assist a clinician in placing apparatus 500 within the intra-articular space such that electrodes 508 contact the targeted area. Targeted area 512 is heated by electrodes 508 in order to thermally weld torn tissue. Thermal welding may be accomplished according to temperature profiles as discussed with respect to apparatus 200 of FIG. 2. In other embodiments, apparatus 500 may be deployed by a hollow needle or trocar device for minimally invasive delivery.

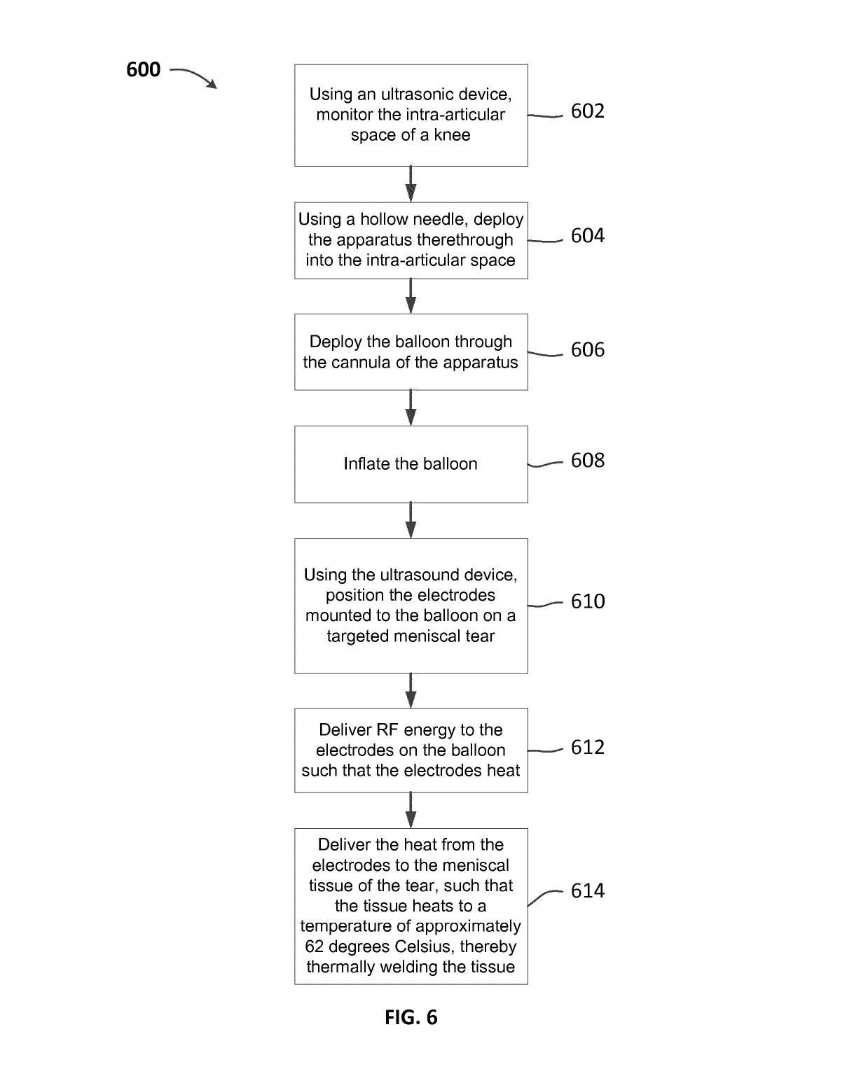

FIG. 6 is a flow diagram illustrating a process 600 for thermally welding torn tissue in accordance with an illustrative embodiment. In alternative embodiments, fewer, additional, and/or different operations may be performed. Also, the use of a flow diagram is not meant to be limiting with respect to the order of operations performed. In an operation 602, an ultrasonic device is used to monitor the intra-articular space of a knee. In an operation 604, a hollow needle is used to deploy an apparatus for thermally treating torn tissue therethrough into the intra-articular space. The apparatus is an apparatus as described herein (e.g., apparatus 200 of FIG. 2, apparatus 300 of FIG. 3, apparatus 400 of FIG. 4, apparatus 500 of FIG. 5, etc.). In another illustrative embodiment, other techniques and arthroscopic visualization may be utilized to monitor the intra-articular space and assist in deployment of the apparatus. In another illustrative embodiment, palpation and anatomic landmark techniques may be used in positioning the apparatus as it is deployed. Palpation and anatomic landmark techniques may be useful in an embodiment where the apparatus is deployed using a trocar device. As an example, such palpation may include the use of 3D computer models of a patient's joint obtained from medical imaging system. A clinician may use the models and landmarks of the joint during palpation as the clinician feels and positions the trocar device.

In an operation 606 and an operation 608, the balloon of the apparatus is deployed through the cannula of the apparatus and is inflated therein. The balloon and cannula are as described herein with reference to FIGS. 1-5 (e.g., cannula 402 and balloon 404 of FIG. 4, cannula 502 and balloon 504 of FIG. 4, etc.). A guidewire may be also used (e.g., guidewire 306 of FIG. 3, guidewire 406 of FIG. 4, etc.) in deploying and providing mechanical support to the balloon. In an illustrative embodiment, the balloon is inflated to substantially conform to the intra-articular space. In another illustrative embodiment, the balloon is smaller than the intra-articular space when inflated so that multiple precise locations in the intra-articular space may be targeted. The size of the balloon may be selected according to the type of procedure being performed (e.g., thermal welding, thermal treatment, etc.).

In an operation 610, the ultrasound device (or other monitoring device) is used generate live images of the intra-articular space which may be used to precisely position the electrodes of the apparatus on a targeted meniscal tear. The guidewire may assist in positioning the electrodes. In an operation 612, energy is delivered to the electrodes via conductive wiring running through the cannula of the apparatus. The electrodes and conductive wiring are as described herein with reference to FIGS. 1-5. In an illustrative embodiment, the amount of energy delivered to the electrodes depends on the desired temperature to be reached in the tissue to be repaired. A computing device may be used to monitor and control the amount of energy provided to the electrodes.

In an operation 614, the energy from the electrodes is delivered to the meniscal tissue of the tear in order to heat the tissue. In an illustrative embodiment, the torn tissue (and surrounding tissue) is heated to a temperature of approximately 62 degrees Celsius. At a temperature of approximately 62 degrees Celsius it is possible to thermally weld together separated tissue. In other embodiments, a desired temperature of the tissue may depend on the type of tissue, or the specific operation being performed. The thermistor of the apparatus (e.g., thermistor 312 of FIG. 3, thermistor 410 of FIG. 4, etc.) may provide temperature feedback, which may be used to monitor the temperature of the tissue being heated. Temperature feedback from the thermistor may be provided to a computing device. The computing device may format the feedback received for use on an electronic display. The computing device may also automatically adjust the energy provided to the electrodes based on the temperature feedback. In one example, as the temperature of the tissue is approaching 62 degrees Celsius, the computing device may automatically cause the amount of energy provided to the electrodes to decrease so that the tissue does not become overheated.

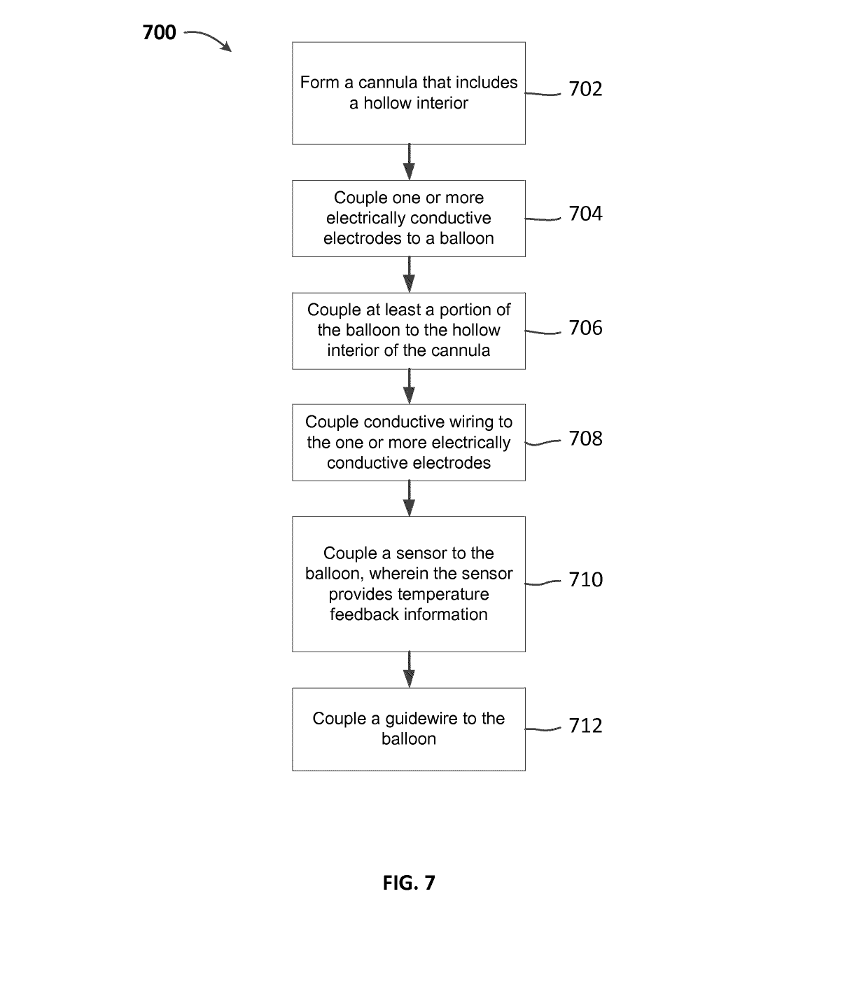

FIG. 7 is a flow diagram illustrating a process 700 for creating an apparatus to treat torn tissue in accordance with an illustrative embodiment. In alternative embodiments, fewer, additional, and/or different operations may be performed. Also, the use of a flow diagram is not meant to be limiting with respect to the order of operations performed. In an operation 702 a cannula is formed that includes a hollow interior. In an operation 704, one or more electrically conductive electrodes are coupled to a balloon. In an operation 706, at least a portion of the balloon is coupled to the hollow interior of the cannula. In an operation 708, conductive wiring is coupled to the one or more electrically conductive electrodes. In an operation 710, a sensor is coupled to the balloon. The sensor is configured to provide temperature feedback information. In one embodiment, the sensor is a thermistor device. In an operation 712, a guidewire is coupled to the balloon. The guidewire may be coupled to an interior portion of the balloon. The gauge and specification of the guidewire may be selected according to the overall size and characteristics of the apparatus being formed. The guidewire may be used to deploy the balloon through the distal end of the hollow interior of the cannula, and further to provide mechanical support to the balloon. In an illustrative embodiment, the cannula is further positioned within or coupled to a hollow needle used for deployment of the cannula. In another embodiment, the cannula may be formed as a component of a trocar device. Other delivery devices known to those skilled in the art are also envisioned.

FIG. 8 is a diagram illustrating a system 800 for thermally treating torn tissue, including an example computing system, arranged in accordance with at least some embodiments presented herein. System 800 includes energy source 802, an apparatus 806 for thermally treating torn tissue, and a computer 810. Energy source 802 includes energy generator 804. Apparatus 806 may be an apparatus for thermally treating torn tissue as described herein (e.g., apparatus 200 of FIG. 2, apparatus 300 of FIG. 3, apparatus 400 of FIG. 4, or apparatus 500 of FIG. 5, etc.). Apparatus 806 includes a temperature sensor 808. In one embodiment, temperature sensor 808 is a thermistor device.

Computer 810 includes a processor 812, memory 814, and may include one or more drives 820. The computer 810 may be implemented as a conventional computer system, an embedded control computer, a laptop, a server computer, a mobile device, a set-top box, a kiosk, a health care information system, a customized machine, or other hardware platform. In one embodiment, computer 810 may be part of a single device also containing energy source 802. In an alternative embodiment, computer 810 may be a standalone device that is in communication with energy source 802. In alternative embodiments, computer 810 may include additional, fewer, and/or different components. Processor 812 can be any type of computer processor known to those of skill in the art, and may be implemented as a general-purpose processor, an application specific integrated circuit (ASIC), one or more field programmable gate arrays (FPGAs), a group of processing components, or other suitable electronic processing components. The processor 812 can be used to receive temperature feedback information from temperature sensor 808, to analyze the temperature feedback information, to execute instructions stored in memory 814, and to generate appropriate signals to control energy source 802, etc. Memory 814 can include any type of computer memory or memories known to those of skill in the art, and can be one or more devices (e.g., RAM, ROM, Flash Memory, hard disk storage, etc.) for storing data and/or computer code for facilitating the various processes described herein. Memory 814 may be or include non-transient volatile memory or non-volatile memory. Memory 814 may include database components, object code components, script components, or any other type of information structure for supporting the various activities and information structures described herein.

The drives 820 and their associated computer storage media may provide storage of computer readable instructions, data structures, program modules and other data for the computer 810. The drives 820 and/or memory 814 can include an operating system 822, application programs 824, program modules 826, and a database 828. Some examples of the program modules 826 may include a user interface, a communications module, and a control parameters module. The control parameters module may include data related to interfacing with energy source 802 and/or apparatus 806. For example, the control parameters module may include information as to how often input should be accepted from temperature sensor 808. As another example, the control parameters module may include information relating to a user's preferences. Memory 814 and drives 820 can each be used to store data obtained from apparatus 806 (e.g., temperature feedback signals from temperature sensor 808, etc.), to store instructions to be executed by processor 812, to store patient information, to store temperature profile information, etc. The computer 810 further includes user input devices 816 and an input through which a user may enter commands and data, and through which data may be received (e.g., from energy source 802 and apparatus 806, etc.). Input devices can include an electronic digitizer, peripheral devices, a microphone, a keyboard and pointing device, commonly referred to as a mouse, trackball or touch pad. Other input devices may include an energy source 802 and an apparatus 806.

These and other input devices can be coupled to the processor 812 through a user input interface that is coupled to a system bus, but may be coupled by other interface and bus structures, such as a parallel port, a serial port, a universal serial bus (“USB”), a FireWire port, or other port. Computers such as the computer 810 may also include other peripheral output devices such as speakers, which may be coupled through an output peripheral interface 818 (via an output) or the like. The output peripheral interface 818 may also be used to communicate with energy source 802 and apparatus 806. As an example, the output may be configured to provide appropriate signals to a graphical display device (e.g. a display that is part of output peripheral interface 818, etc.). Such signals may correspond to characteristics of the temperatures of tissue or electrodes, or of characteristics of energy that is provided to apparatus 806 from energy source 802. In one embodiment, the input and output are coupled to a separate LCD display of output peripheral interface 818, and signals are sent to the LCD display to show the temperature of damaged tissue as it is being heated by apparatus 806. The input and output can operate via wired or wireless communication according to any protocol(s) known to those of skill in the art. The input and output can receive data from apparatus 806, and processor 812 can be used to form images or graphical data based on the received data. The output can also be used to provide instructions to energy source 802 such that a clinician (or other operator) can use an a user input device 816 and output peripheral interface 818 to control energy source 802 and in turn adjust energy provided to apparatus 806.

The computer 810 may operate in a networked environment using logical connections to one or more computers or devices, such as a remote computer or device (e.g., energy source 802 and apparatus 806) coupled to a network interface 830. As an example, the remote computer may be a personal computer, a server, a router, a network PC, a peer device or other common network node, and can include many or all of the elements described above relative to the computer 810. Networking environments are commonplace in health care organizations, enterprise-wide area networks (“WAN”), local area networks (“LAN”), wireless networks, intranets, and the Internet. When used in a networking environment, the computer 810 may be coupled to the network through the network interface 830 or an adapter. When used in a WAN networking environment, the computer 810 typically includes a modem or other means for establishing communications over the WAN, such as the Internet or the network 832. The WAN may include the Internet, the illustrated network 832, various other networks, or any combination thereof. It will be appreciated that other mechanisms of establishing a communications link, ring, mesh, bus, cloud, or network between the computers may be used.

In an illustrative embodiment, user input devices 816 include an ultrasonic transceiver device (e.g. a portable or fixed ultrasound device, an ultrasonic transducer, etc.) The ultrasonic transceiver device may provide ultrasonic information based on an intra-articular space into which apparatus 806 is inserted. The ultrasonic information may be provided according to any protocol(s) known to those of skill in the art, and may be transmitted to computer 810 via an input. Computer 810 may receive the ultrasonic information and format the information for use by a display coupled to output peripheral interface 818. For example, processor 812 may generate appropriate signals such that received ultrasonic information is displayed (e.g., via output peripheral interface 818, etc.) as real time images of the intra-articular space. Such real time images may be used by a clinician to aid in positioning apparatus 806 within the intra-articular space. In one embodiment, computer 810 may be part of a single device also containing an ultrasonic transceiver device. In an alternative embodiment, computer 810 may be a standalone device that is in communication with an ultrasonic transceiver device.

In an illustrative embodiment, a clinician inserts apparatus 806 into the intra-articular space of a patient's knee. The clinician deploys and inflates the balloon of apparatus 806, and positions the electrodes of apparatus 806 in close proximity to a tear in the patient's meniscal tissue. Positioning of the apparatus may be facilitated by use of an ultrasonic transceiver device as discussed above. The clinician can enter commands via a user input device 816 to cause energy source 802 to supply energy to the electrodes of apparatus 806. Processor 812 receives the input commands (e.g., through a touchscreen input, a mouse, a keyboard, etc.) and generates an appropriate control signal. The control signal is configured to control characteristics of the energy provided to the electrodes of apparatus 806. The control signal may cause adjustments to the energy signal amplitude, frequency, modulation, etc. The control signal is transmitted to energy source 802, which generates an energy signal as specified by the control signal. In one embodiment, the energy signal is a radiofrequency energy signal and energy generator 804 is a radiofrequency energy generator. Energy generator 804 includes components utilized for signal generation (e.g., power supply, AC to DC transformers, etc.) as known to those skilled in the art. Energy source 802 further includes appropriate components for controlling and adjusting the energy signal (e.g., modulators, regulators, etc.) as known to those skilled in the art. As an example, the control signal may cause energy source 802 to increase or decrease the amplitude of a generated radiofrequency signal. In another example, the control signal may cause energy source 802 to start or stop the transmission of radiofrequency energy to the electrodes of apparatus 806.

Transmission of energy to the electrodes of apparatus 806 may be implemented via conducting wires (e.g., conducting wiring 308 of FIG. 3) coupled to an output of energy source 802 and the electrodes of apparatus 806. Temperature sensor 808 provides sensed temperature information as a temperature feedback signal sent to computer 810. Computer 810 may monitor the temperature feedback signal and adjust the control signal according to a desired temperature or heating profile. In one embodiment, memory 814 or drives 820 contain instructions to automatically generate a control signal such that a tissue temperature of approximately 62 degrees Celsius is maintained for a certain amount of time. Further instructions may also exist to disallow the tissue temperature to exceed 69 degrees Celsius. In one embodiment, the desired tissue temperature or heating profile is input via a user input device 816 by a clinician, and a corresponding control signal is generated by computer 810.

In one embodiment, energy source 802 also provides energy source feedback signals to computer 810. The energy source feedback signals include information related to the type of signal output by energy source 802, and may be used by computer 810 in maintaining a certain temperature profile in tissue. The energy feedback signals may also include status information related to the components of energy source 802. As an example, such status information may be used by computer 810 to detect component failures, etc.

Any of the operations described herein can be performed by computer-readable (or computer-executable) instructions that are stored on a computer-readable medium such as memory 814 or as included in drives 820. The computer-readable medium can be a computer memory, database, or other storage medium that is capable of storing such instructions. Upon execution of the computer-readable instructions by a computing device such as computer 810, the instructions can cause the computing device to perform the operations described herein.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

1. An apparatus for thermally treating torn tissue, the apparatus comprising:
a cannula comprising a hollow interior that is configured to receive a fluid;
a balloon, wherein at least a portion of the balloon is positioned within the hollow interior of the cannula, and wherein the fluid received through the hollow interior of the cannula inflates the balloon; and
one or more electrically conductive electrodes mounted to the balloon, wherein the one or more electrically conductive electrodes are configured to deliver heat to tissue.

2. (canceled)

3. The apparatus of claim 1, further comprising conductive wiring coupled to the one or more electrically conductive electrodes, wherein the conductive wiring is configured to provide energy to heat the one or more electrically conductive electrodes.

4.-6. (canceled)

7. The apparatus of claim 1, further comprising a guidewire configured to:
deploy the balloon through a distal end of the hollow interior member; and
provide mechanical support to the balloon.

8. The apparatus of claim 1, further comprising a sensor, wherein the sensor is configured to provide temperature feedback information.

9. The apparatus of claim 8, wherein the sensor is a thermistor device.

10. The apparatus of claim 1, wherein the one or more electrically conductive electrodes are sized and positioned to heat a specific area with a specific temperature gradient.

11. The apparatus of claim 1, further comprising a hollow needle configured to deliver the cannula to a space proximate to the tissue.

12. The apparatus of claim 1, further comprising a trocar device configured to deliver the cannula to a space proximate to the tissue.

13. A method of thermally welding torn tissue, the method comprising:
inserting at least a portion of a cannula into an intra-articular space, wherein the cannula comprises a hollow interior that includes at least a portion of a balloon;
inflating the balloon within the intra-articular space such that one or more electrically conductive electrodes mounted to the balloon contact tissue; and
delivering heat to the tissue through the one or more electrically conductive electrodes.

14. (canceled)

15. The method of claim 13, further comprising using a guidewire to deploy the balloon through a distal end of the hollow interior and into the intra-articular space.

16. (canceled)

17. The method of claim 13, further comprising receiving temperature feedback information from a sensor coupled to the balloon.

18. The method of claim 17, further comprising adjusting an amount of energy provided to the one or more electrically conductive electrodes based on the temperature feedback information such that the tissue is heated to a temperature that is less than or equal to 69 degrees Celsius.

19.-21. (canceled)

22. The method of claim 13, further comprising positioning the balloon within the intra-articular space using ultrasound.

23. A method of creating an apparatus to treat torn tissue, the method comprising:
forming a cannula that includes a hollow interior;
coupling one or more electrically conductive electrodes to a balloon;
coupling at least a portion of the balloon to the hollow interior of the cannula; and
coupling conductive wiring to the one or more electrically conductive electrodes.

24. The method of claim 23, further comprising coupling a sensor to the balloon, wherein the sensor provides temperature feedback information.

25. (canceled)

26. The method of claim 23, further comprising coupling a guidewire to the balloon, wherein the guidewire deploys the balloon through a distal end of the hollow interior of the cannula, and wherein the guidewire provides mechanical support to the balloon.

27. (canceled)

28. (canceled)

29. A system, comprising:
an apparatus to treat torn tissue comprising:
a cannula having a hollow interior;
a balloon configured to be deployed through a distal end of the hollow interior of the cannula, wherein at least a portion of the balloon is positioned within the hollow interior of the cannula;
one or more electrically conductive electrodes coupled to the balloon and configured to deliver heat to tissue; and
a sensor coupled to the one or more electrically conductive electrodes; and
a computing device comprising:
a memory configured to receive and store temperature feedback information from the sensor; and
a processor operatively coupled to the memory and configured to control heat output of the one or more electrically conductive electrodes based on the temperature feedback information.

30. (canceled)

31. The system of claim 29, further comprising a guidewire configured to:
deploy the balloon through the distal end of the hollow interior of the cannula; and
provide mechanical support to the balloon.

32.-35. (canceled)

36. The system of claim 29, wherein the computing device further comprises a display, and wherein the display is configured to present images based on ultrasonic information regarding an intra-articular space into which the cannula is inserted.

37. The system of claim 29, wherein the computing device further comprises a display, and wherein the display is configured to present images based on the temperature feedback information from the sensor.

38. (canceled)

39. (canceled)
